(19) United States
(12) Patent Application Publication
(10) Pub. No.: US 2009/0143601 A1
(43) Pub. Date: Jun. 4, 2009

(54) METHOD FOR PREVENTING FUMARIC ACID DEPOSITS IN THE PRODUCTION OF MALEIC ACID ANHYDRIDE

(75) Inventors: Rolf Pinkos, Bad Durkheim (DE); Gerd Kaibel, Lampertheim (DE); Ellen Dahlhoff, Limburgerhof (DE); Gunther Windecker, Ludwigshafen (DE); Jens Heimann, Worms (DE)

Correspondence Address: CONNOLLY BOVE LODGE & HUTZ LLP, 1875 EYE STREET, N.W., SUITE 1100, WASHINGTON, DC, 20006, US

(73) Assignee: BASF SE, LUDWIGSHAFEN (DE)

(21) Appl. No.: 12/300,571

(22) PCT Filed: May 16, 2007
(86) PCT No.: PCT/EP07/54788

§ 371 Date: Nov. 12, 2008

(30) Foreign Application Priority Data
May 24, 2006 (DE) 102006024903.8

(51) Int. Cl. C07D 307/60

(52) U.S. Cl. 549/262

(57) ABSTRACT

The present invention provides a process for preventing fumaric acid deposits in the preparation of maleic anhydride, comprising the following steps:

a) absorption of a C₄-dicarboxylic acid or of a derivative from a crude product mixture into an organic solvent or water as an absorbent,

b) removal of the C₄-dicarboxylic acid or of a derivative from the absorbent,

the absorbent thus recovered being catalytically hydrogenated fully or partly and recycled fully or partly into the absorption stage (a).

The present invention relates to processes for preventing fumaric acid deposits in the preparation of maleic anhydride (MA), in which MA is absorbed from a crude product mixture into an organic solvent or water as an absorbent, then MA is removed from the absorbent and the absorbent thus recovered or a substream thereof is catalytically hydrogenated and recycled fully or partly into the absorption stage (a).

The process according to the invention serves to improve the industrial scale preparation of maleic anhydride. Maleic anhydride is a valuable starting material, a base substance for polymers or serves, via the hydrogenation of MA via the succinic anhydride (SA) intermediate, for the preparation of gamma-butyrolactone (GBL), butanediol (BDO) and tetrahydrofuran (THF).

Maleic anhydride can be obtained by partial oxidation of hydrocarbons such as butane or benzene. The valuable product is typically absorbed from the maleic anhydride-containing offgas of the partial oxidation in a solvent. This absorbs not only MA but also further components present in the oxidation offgas, for example water. Water reacts with the maleic anhydride to give maleic acid which in turn isomerizes to fumaric acid. Fumaric acid is a diacid which is very sparingly soluble in water or organic solvents and forms deposits and, as a result, can block plant parts, for example columns, heat exchangers, pumps, tubes and the like. In order to prevent such blockages caused by fumaric acid, there are already proposals in the prior art.

For instance, WO 96/29323 describes the washing of the fumaric acid-comprising absorbent with an aqueous extractant, in order thus to prevent deposits. A disadvantage of this process is the high complexity which is needed to mix the wash water into an industrial scale plant for preparing C₄-dicarboxylic acids or derivatives thereof and to separate the phases again. In addition, the unavoidable loss of valuable product and solvent makes the process uneconomic. Moreover, the additional water input into the process enhances the fumaric acid formation further.

Proceeding from this state of the art, it is an object of the invention to prevent fumaric acid deposits on plant parts and blockages, deinstallation and cleaning operations and shutdowns caused thereby in the process for preparing C₄-dicarboxylic acids and/or derivatives thereof.

This object is achieved by a process for preventing fumaric acid deposits in the preparation of maleic anhydride, comprising the following steps:
a) absorption of maleic anhydride from a crude product mixture into an organic solvent or water as an absorbent,
b) removal of the maleic anhydride from the absorbent,
   the absorbent.thus recovered being catalytically hydrogenated fully or partly and recycled fully or partly into the absorption stage (a). Preference is given to hydrogenating a substream and to recycling it fully into the absorption stage (a).

The process according to the invention avoids the aforementioned disadvantages by Hydrogenating fumaric acid present in the absorbent over a hydrogenation catalyst with hydrogen to give succinic acid. Surprisingly, even in the presence of solid fumaric acid, high selectivities are achieved at low pressures and small amounts of catalyst to be expended. Even fumaric acid deposits already formed in pipelines or other plant parts are removed again.

The inventive hydrogenation process may comprise a preceding stage which comprises the preparation of MA by partial oxidation of a suitable hydrocarbon. Suitable hydrocarbons are benzene, C₄-olefins (e.g. n-butenes, C₄-raffinate streams) or n-butane. Particular preference is given to using n-butane, since it is an inexpensive, economically viable feedstock. Processes for the partial oxidation of n-butane are described, for example, in Ullmann's Encyclopedia of Industrial Chemistry, 6^{th} Edition, Electronic Release, Maleic and Fumaric Acids—Maleic Anhydride.

The reaction effluent thus obtained, the crude product mixture, is then taken up in water or preferably in a suitable organic solvent as an absorbent, or mixture thereof, the organic solvent preferably having a boiling point at least 30° C. higher than MA at atmospheric pressure.

The maleic anhydride-comprising gas stream from the partial oxidation can be contacted with the solvent (absorbent) in one or more absorption stages in various ways at pressures (absolute) of from 0.8 to 10 bar and temperatures of 50-300° C.: (i) introduction of the gas stream into the solvent (for example via gas introduction nozzles or sparging rings), (ii) spraying the solvent into the gas stream and (iii) countercurrent contact between the gas stream flowing upward and the solvent flowing downward in a tray column or column with structured packing. In all three variants, the apparatus for gas absorption known to those skilled in the art can be used. VVhen selecting the solvent (absorbent) to be used, it should be ensured, especially in the case of MA recovery, that it does not react with the reactant, the MA used. Suitable absorbents are: tricresyl phosphate, dibutyl maleate, butyl maleate, high molecular weight waxes, aromatic hydrocarbons having a molecular weight between 150 and 400 and a boiling point above 140° C., for example dibenzylbenzene; alkyl phthalates and dialkyl phthalates having C₁-C₁₈-alkyl groups, for example dimethyl phthalate, diethyl phthalate, dibutyl phthalate, di-n-propyl and diisopropyl phthalate, undecyl phthalate, diundecyl phthalate, methyl phthalate, ethyl phthalate, butyl phthalate, n-propyl or isopropyl phthalate; di-C₁-C₄-alkyl esters of other aromatic and aliphatic dicarboxylic acids, for example dimethyl-2,3-naphthalenedicarboxylic acid dimethyl ester, dimethyl-1,4-cyclohexanedicarboxylic acid dimethyl ester; C₁-C₄-alkyl esters of other aromatic and aliphatic dicarboxylic acids, for example dimethyl 2,3-naphthalenedicarboxylate, dimethyl 1,4-cyclohexanedicarboxylate of long-chain fatty acids having, for example, from 14 to 30 carbon atoms, high-boiling ethers, for example dimethyl ethers of polyethylene glycol, for example tetraethylene glycol dimethyl ether.

The use of phthalates is preferred.

The solution resulting from the treatment with the absorbent generally has an MA content of from about 5 to 400 grams per liter.

The offgas stream which remains after the treatment with the absorbent comprises, in addition to water, mainly the by-products of the preceding partial oxidation, such as carbon monoxide, carbon dioxide, unconverted butanes, acetic acid, and acrylic acid. The offgas stream is virtually free of MA.

Subsequently, the dissolved MA is driven out of or removed from the absorbent. This can be done with hydrogen at or not more than 10% above the pressure of a subsequent hydrogenation of the MA to THF, BDO or GBL, preferably at from 100 to 250° C. and pressures (absolute) of from 0.8 to 30 bar. In the stripping column, a temperature profile is observed which arises from the boiling points of MA at the top and the virtually MA-free absorbent at the bottom of the column at the particular column pressure and the set dilution with carrier gas (in the first case with hydrogen). In order to prevent losses of solvent, rectifying internals may be present above the feed of the crude MA stream.

Alternatively to the preferred hydrogen stripping, the MA dissolved in the absorbent can be removed in a distillation unit at pressures of generally from 0.01 to 5 bar and temperatures of from 65 to 300° C. The distillation can be performed in one stage or a plurality of stages, for example in separating apparatus having one stage or a plurality of stages, for example columns with a plurality of separating stages, for example rectifying columns, columns with random packing, bubble-cap tray columns or columns with structured packing.

The recovered virtually MA-free absorbent which has been drawn off from the bottom of the distillation unit or of the stripping column is now fed to the inventive hydrogenation and hydrogenated over a hydrogenation catalyst preferably at temperatures between 20 and 300° C., more preferably from 60 to 270° C. and especially preferably from 100 to 250° C., and at pressures (absolute values) of preferably from 0.1 to 300 bar, more preferably from 0.5 to 50 bar, especially preferably from 0.8 to 20 bar.

The contents of fumaric acid in the absorbent recovered in stage b) (total amount of fumaric acid present in homogeneously dissolved and suspended form) before the inventive hydrogenation stage is typically from 0.01 to 5% by weight. Usually, the recovered absorbent has a content between 0.02 and 2% by weight of fumaric acid. The molar amount of hydrogen for the inventive hydrogenation stage is generally selected such that at least one mole of hydrogen is present per mole of fumaric acid. However, an excess of hydrogen is uncritical. The hydrogen may be dissolved; gaseous hydrogen may additionally be present. After the inventive hydrogenation stage, the fumaric acid content is generally below 0.1% by weight, preferably below 0.05% by weight. The fumaric acid content should only be so high that a homogeneous solution is present at a given temperature.

The process according to the invention can be performed batchwise, semicontinuously or continuously. Continuous performance is preferred.

The hydrogenation is effected in the liquid phase over heterogeneous catalysts which may be arranged in a fixed manner or suspended, preference being given to fixed catalysts (fixed bed catalysts).

The usable catalysts preferably comprise at least one metal from group 7, 8, 9, 10 or 11 of the Periodic Table of the Elements or compounds thereof, for example oxides. The catalysts usable in accordance with the invention more preferably comprise at least one element selected from the group consisting of Re, Fe, Ru, Co, Rh, Ir, Ni, Pd, Pt, Cu and Au. Especially preferably, the catalysts usable in accordance with the invention comprise at least one element selected from the group consisting of Ni, Pd, Pt, Ru and Cu. Moreover, the catalysts usable in accordance with the invention especially preferably comprise Pd, Pt, Ru or Ni.

In particular, at least one heterogeneous catalyst is suitable, in which case at least one of the abovementioned metals (active metals) may be used as a metal as such, as a Raney catalyst and/or applied to a customary support. When two or more active metals are used, they may be present separately or as an alloy. It is possible in this context to use at least one metal as such and at least one other metal as a Raney catalyst, or at least one metal as such and at least one other metal applied to at least one support, or at least one metal as a Raney catalyst and at least one other metal applied to at least one support, or at least one metal as such and at least one other metal as a Raney catalyst and at least one other metal applied to at least one support.

The catalysts used may, for example, also be precipitation catalysts. Such catalysts may be prepared by precipitating their catalytically active components from their salt solutions, especially from the solutions of their nitrates and/or acetates, for example by adding solutions of alkali metal and/or alkaline earth metal hydroxide and/or alkali metal and/or alkaline earth metal carbonate solutions, for example sparingly soluble hydroxides, oxide hydrates, basic salts or carbonates, then drying the resulting precipitates and then converting them by calcination at generally from 300 to 700° C., in particular from 400 to 600° C., to the corresponding oxides, mixed oxides and/or mixed-valency oxides, which are reduced to the metals in question and/or oxidic compounds of lower oxidation state and converted to the actual catalytically active form by a treatment with hydrogen or with hydrogen-comprising gases in the range from generally 50 to 700° C., in particular from 100 to 400° C. Reduction is effected generally until no further water is formed. In the preparation of precipitation catalysts which comprise a support material, the catalytically active components can be precipitated in the presence of the support material in question. The catalytically active components can advantageously be precipitated from the salt solutions in question simultaneously with the support material.

Preference is given to using hydrogenation catalysts which comprise the metals or metal compounds which catalyze the hydrogenation deposited on a support material.

Apart from the abovementioned precipitation catalysts which, apart from the catalytically active components, additionally comprise a support material, suitable support materials for the process according to the invention are generally those in which the catalytically hydrogenating component has been applied to a support material, for exarnple, by impregnation.

The way in which the catalytically active metal is applied to the support is generally not critical, and it can:be accomplished in various ways. The catalytically active metals can be applied to these support materials, for example, by impregnation with solutions or suspensions of the salts or oxides of the elements in question, drying and subsequent reduction of the metal compounds to the metals in question or compounds of lower oxidation state by means of a reducing agent, preferably with hydrogen or complex hydrides. Another means of applying the catalytically active metals to these supports consists in impregnating the supports with solutions of thermally readily decomposable salts, for example with nitrates, or thermally readily decomposable complexes, for example carbonyl or hydrido complexes, of the catalytically active metals, and in heating the support thus impregnated to temperatures in the range from 300 to 600° C. to thermally decompose the adsorbed metal compounds. This thermal decomposition is preferably undertaken under a protective gas atmosphere. Suitable protective gases are, for example, nitrogen, carbon dioxide, hydrogen or the noble gases. In addition, the catalytically active metals can be deposited on the catalyst support by vapor deposition or by flame spraying. The content of the catalytically active metals in these supported catalysts is in principle uncritical for the success of the process according to the invention. In general, relatively high contents of catalytically active metals in these supported catalysts lead to higher space-time yields than relatively low contents. In general, supported catalysts whose content of catalytically active metals is in the range from 0.01 to 90% by weight, preferably in the range from 0.1 to 40% by weight, based on the total weight of the catalyst, are used. Since these contents are based on the entire catalyst including support material, but the different support materials have very different specific weights and specific surface areas, it is also conceivable that the contents may also be lower or higher than these contents without this having a disadvantageous effect on the result of the process according to the invention. It will be appreciated that it is also possible for a plurality of the catalytically active metals to be applied on the particular support material. Moreover, the catalytically active metals may be applied to the support, for example, by the process of DE-A 25 19 817, EP-A 1 477 219 or EP-A 0 285 420. In the catalysts according to the aforementioned documents, the catalytically active metals are present in the form of alloys which are obtained by thermal treatment and/or reduction of the, for example, by impregnation of the support material with a salt or complex of the aforementioned metals.

Owing to the toxicity of chromium catalysts, preference is given to using chromium-free catalysts. It will be appreciated that corresponding chromium catalysts known to those skilled in the art are also suitable industrially for use in the process according to the invention, which, however, does not give rise to the desired advantages, which are especially of environmental and occupational safety nature. Both the precipitation catalysts and the supported catalysts can also be activated in situ at the start of the reaction by the hydrogen present. Preference is given to activating these catalysts separately before use.

Both for precipitation catalysts and for supported catalysts, the support materials used may be the oxides of aluminum and of titanium, zirconium dioxide, silicon dioxide, aluminas, for example montmorillonites, bentonites, silicates, for example magnesium or aluminum silicates, zeolites, for example of the ZSM-5 or ZSM-10 structure types, or activated carbon. Preferred support materials are aluminum oxides, titanium dioxides, silicon dioxide, zirconium dioxide and activated carbon. It will be appreciated that mixtures of different support materials can also serve as the support for catalysts usable in the process according to the invention. Also suitable are metallic supports on which the hydrogenation-active metal has been deposited, for example Cu on which, for example, Pd, Pt or Ru has been deposited from the corresponding metal salts dissolved in water.

Especially preferred inventive catalysts are supported catalysts which comprise Ni, Pt and/or Pd, especially preferred supports being activated carbon, aluminum oxide, titanium dioxide; and/or silicon dioxide or mixtures thereof.

A heterogeneous catalyst usable in accordance with the invention may be used in the process according to the invention as a suspension catalyst and/or as a fixed bed catalyst.

The inventive hydrogenation stage is preferably performed in one or more separate reactors. In a preferred embodiment, the separate hydrogenation reactor for the hydrogenation stage of the process according to the invention is fed with offgas hydrogen from MA hydrogenation to give SA, GBL, THF and/or BDO.

However, the inventive hydrogenation can also be effected within the stripping column for removing the MA from the absorbent. In this particular embodiment, the stripping column has, preferably in the lower section where the MA concentration is already below 1% by weight, a fixed bed catalyst, for example in the form of catalytic packing.

When, in the process according to the invention, the hydrogenation stage is performed with at least one suspension catalyst, preference is given to hydrogenating in at least one stirred reactor, at least one bubble column and/or a packed bubble column, or in a combination of two or more identical or different reactors.

The term “different reactors” denotes either different reactor types or reactors of the same type which differ, for example, by their geometry, for example their volume and/or their cross section, and/or by the hydrogenation conditions in the reactors.

When, for example, in the process according to the invention, the hydrogenation is performed with at least one fixed bed catalyst, preference is given to using at least one tubular reactor, for example at least one shaft reactor and/or at least one tube bundle reactor, an individual reactor being operable in liquid-phase or trickle mode. When two or more reactors are used, at least one can be operated in liquid-phase mode and at least one in trickle mode.

When the catalyst used in the hydrogenation stage of the process according to the invention is a heterogeneous catalyst as a suspension catalyst, it is preferably removed after the hydrogenation by at least one filtration step. The catalyst removed in this way may be recycled into the hydrogenation stage.

The heat released in the hydrogenation is generally not removed. Should this, however, be necessary, it can be removed in the reactor used in accordance with the invention internally, for example via cooling coils, and/or externally, for example via at least one heat.exchanger.

When the hydrogenation: is performed over at least one suspended catalyst, the residence time is generally in the range from 0.01 to 10 h, for example in the range from 0.5 to 5 h, preferably in the range from 0.5 to 2 h and more preferably in the range from 0.1 to 1 h. It is unimportant whether, in accordance with the invention, a main reactor and a postreactor or additionally further reactors are used. For all of these embodiments, the total residence time is within the above-specified ranges.

When, in the process according to the invention, the inventive hydrogenation stage is performed in continuous mode over at least one fixed bed catalyst, the catalyst hourly space velocity (kg of feed/liters of catalyst×h) is generally in the range from 0.05 to 1000, preferably in the range from 0.1 to 500 and more preferably in the range from 0.5 to 100. It is unimportant whether, in accordance with the invention, a main reactor and a postreactor or additionally further reactors are used. For all of these embodiments, the total hourly space velocity is within the above-specified ranges. Feed should be understood to mean the fumaric acid-containing recovered absorbent.

Further components present in the feed are in particular those which are likewise absorbed by the solvent in the absorption stage. Examples include maleic acid, MA, alkyl-substituted maleic acid derivatives, acrylic acid, methacrylic acid and acetic acid. In addition, hydrogenation products of the process according to the invention, such as succinic acid and succinic anhydride, are present. Further additional compounds are those which can form from the solvent, these depending upon the nature of the solvent. When, for example, phthalates are used, not only phthalic anhydride and its monoesters but likewise the esters of the aforementioned acids are possible.

To remove high-boiling components, for example succinic acid, it is possible in a particular embodiment to subject a substream of the absorbent recovered to a distillation after the hydrogenation stage and before recycling into the absorption stage a).

In addition, the succinic acid formed by the inventive hydrogenation can be removed as such or as succinic anhydride from the absorbent by measures known to those skilled in the art, for example by partial condensation, condensation, distillation and stripping, analogously to the aforementioned MA stripping.

The process according to the invention will now be illustrated in detail in the examples which follow.

### EXAMPLES

### Comparative Example 1

1a) Experimental Apparatus

The apparatus used consisted of a feed for the MA melt, a water feed upstream of the circulation pump, a distillation column with bottom heating and reflux divider for removing the. maleic anhydride (MA) between two heat exchangers, and a pressure regulator. Owing to trace heating, the temperature in all plant parts was at least 70° C.

A circulation stream of 3 l/h of dibutyl phthalate at 1.2 bar upstream of the distillation column was heated to 200° C. and conducted into the distillation column downstream of the pressure regulator. At a column pressure of 0.2 bar absolute, the bottom temperature was 230° C. After the circulation stream withdrawn via a bottom outlet had been cooled to 90° C., approx. 0.3 kg of MA/h in the form of a melt and approx. 15 g of water/h were metered into the circulation. MA and the majority of the water were distilled off via the top of the column at a reflux ratio of 1 (distillate). The distillate consisted of predominantly MA and small amounts of water and maleic acid. The apparatus was operated further over a period of 4 days. With daily sampling, a visual test and a gas chromatography analysis showed that suspended solid had formed in the plant in the form of fumaric acid. After 4 days, the pressure regulator became blocked and the plant shut down. After the circulation system had been emptied, fumaric acid deposits were found in the pipelines and the heat exchangers.

### Example 2

2a) Experimental Apparatus

The apparatus 2a) used differs from the experimental apparatus 1a) from comparative example 1 by a 10 ml oil-heated or -cooled tubular reactor which had been installed downstream of the bottom outlet of the column and upstream of the heat exchanger connected upstream of the column, had been filled with 3 mm extrudates of a catalyst consisting of 5% by weight of palladium on activated carbon and was flowed through in trickle mode.

Under otherwise identical experimental conditions and quantitative ratios, in a departure from comparative example 1, a hydrogen stream of approx. 0.5 liter/h was metered into the reactor at a temperature of approx. 180° C. The plant was operated for 10 days. Within this time, there was no blockage. The daily samples showed no solids in the visual test and gas chromatography analysis. Also found in the distillate as well as MA were succinic anhydride, maleic acid and succinic acid.

### Inventive Examples 3 to 5

Inventive example 2 was repeated, except that, instead of Pd on activated carbon, 0.15% by weight of palladium on aluminum oxide was used in example 3, 10% by weight of Ni on activated carbon in example 4, and 0.15% by weight of platinum on aluminum oxide in example 5. In all examples, the same effect as in inventive example 2 was obtained.

### Example 6

Inventive example 2 was repeated, except that no hydrogen was fed into the reactor for the first 3 days. Samples from the circulation system had a solids content of fumaric acid after 2 days. After the 3rd day, as in inventive example 2, hydrogen was metered in in an amount of approx. 0.5 liter/h. When another sample was taken on the next day, no fumaric acid could be detected any longer by a visual test or gas chromatography analysis.

This example shows that, under the inventive conditions, once fumaric acid has been formed and has already been deposited, it can likewise be hydrogenated.

### Comparative Example 7

7a) Experimental Apparatus

The pressure apparatus used consists of a feed for the MA melt and a water feed upstream of a circulation pump and a stripping column (stripper) operated with hydrogen to remove the MA between two heat exchangers, and a pressure regulator. Owing to trace heating, the temperature in all plant parts was at least 70° C.

A circulation stream of 1.5 l/h of dibutyl phthalate was introduced into the stripper at 200° C. via a pressure regulator (approx. 10 bar absolute). 1.5 m³ (STP)/h of hydrogen were introduced at a temperature of approx. 150° C. at 9 bar (absolute) into the bottom of the stripper filled with 5 mm Raschig rings. After the circulation stream withdrawn from the stripper via a bottom outlet had been cooled to 90° C., 0.1 kg of MA/h in the form of a melt and approx. 5 g of water/h were metered into the circulation system. The top product of the stripper (hydrogen, MA and small amounts of dibutyl phthalate) was hydrogenated to THF in a manner known per se over a Cu/aluminum oxide catalyst at 230-260° C. and 9 bar in a tubular reactor.

After 6 days of operating time, the plant shut down owing to a blockage in the heat exchanger downstream of the bottom outlet of the stripper. After the circulation system had been emptied, fumaric acid deposits were found in the pipelines and the heat. exchangers.

### Example 8

8a) Experimental Apparatus

The pressure apparatus 8a) used differs from the experimental apparatus 7a) from comparative example 7 in that the Raschig rings in the lower region of the stripper were replaced by 50 ml of 0.15% by weight of palladium on aluminum oxide. Under otherwise identical experimental conditions and quantitative ratios to those in comparative example 7, the plant was operated for 10 days. Within this time, there was no blockage. The daily samples showed no solids. The subsequent hydrogenation to THF was not impaired by the hydrogenation of the fumaric acid in the circulation of the dibutyl phthalate, i.e. succinic acid and succinic anhydride caused no disruption.

1. A process for preventing fumaric acid deposits in the preparation of maleic anhydride, comprising:
a) absorption of maleic anhydride from a crude product mixture into an organic solvent or water as an absorbent,
b) removal of the maleic anhydride from the absorbent, the absorbent thus recovered being catalytically hydrogenated fully or partly and recycled fully or partly into the absorption stage (a).

2. The process according to claim 1, wherein succinic acid is removed from the absorbent recovered in step b) after the hydrogenation.

3. The process according to claim 1, wherein a substream of the recovered absorbent is hydrogenated and recycled fully.

4. The process according to claim 1, wherein a hydrogenation catalyst comprising at least one of the metals of group 7, 8, 9, 10 or 11 of the Periodic Table of the Elements is used.

5. The process according to claim 1, wherein hydrogenation is effected at a temperature of from 20° C. to 300° C. and a pressure of from 0.1 to 300 bar.

6. The process according to claim 1, wherein at least one hydrogenation reactor selected from the group consisting of tubular reactors, shaft reactors, reactors with internal heat removal, tube bundle reactors and fluidized bed reactors is used.

7. The process according to claim 1, wherein a plurality of reactors are used in parallel or in series connection in the hydrogenation stage.

8. The process according to claim 1, wherein the support material of the hydrogenation catalyst is selected from the oxides of aluminum and of titanium, zirconium dioxide, silicon dioxide, aluminas, montmorillonites, bentonites, silicates, zeolites, activated carbon or mixture thereof.

9. The process according to claim 1, wherein the hydrogenation catalyst comprises one or more further metals selected from Re, Fe, Ru, Rh, Ir, Ni, Pd, Pt, Cu and Au, or compounds thereof.

10. The process according to claim 1, wherein the hydrogenation catalyst is used in the form of shaped bodies.

11. The process according to claim 1, wherein maleic anhydride-comprising crude product mixture which has been obtained by oxidizing benzene, C₄-olefins or n-butane is used.

12. The process according to claim 1, wherein the maleic anhydride in stage b) is driven out of the absorbent with hydrogen.

13. The process according to laim 1, wherein the maleic anhydride in stage b) is removed by distillation from the absorbent.

14. The process according to claim 1, wherein the absorbent is selected from the group consisting of tricresyl phosphate, dibutyl maleate, high molecular weight waxes, aromatic hydrocarbons having a molecular weight between 150 and 400 and a boiling point above 140° C., methyl esters of long-chain fatty acids having from 14 to 30 carbon atoms, high-boiling ethers, and alkyl phthalates and dialkyl phthalates with C₁-C₁₈-alkyl groups.

15. The process according to claim 1, wherein the maleic anhydride is driven out of the absorbent under reduced pressure or at pressures which correspond to the pressure of the hydrogenation or are a maximum of 10% above this pressure.

16. The process according to claim 1, wherein the process is performed batchwise, semicontinuously or continuously.

17. The process according to claim 1, wherein the SA is removed by partial condensation, condensation, stripping or distillation.

18. The process according to claim 1, wherein the hydrogenation catalyst is used in the form of extrudates, ribbed extrudates, tablets, rings, spheres or spall.

19. The process according to claim 1, wherein the process is performed continuously.
